(19) United States
(12) Patent Application Publication
(10) Pub. No.: US 2016/0040209 A1
(43) Pub. Date: Feb. 11, 2016

(54) FAST QUANTIFICATION OF ENZYME ACTIVITY BY ELECTROANALYSIS

(71) Applicant: BOARD OF REGENTS, THE UNIVERSITY OF TEXAS SYSTEM, Austin, TX (US)

(72) Inventors: Maogen Zhang, San Antonio, TX (US); Waldemar Gorski, San Antonio, TX (US)

(21) Appl. No.: 14/782,088

(22) PCT Filed: Apr. 2, 2014
(86) PCT No.: PCT/US14/32713

§ 371 Date: Oct. 2, 2015

Related U.S. Application Data
(60) Provisional application No. 61/807,571, filed on Apr. 2, 2013.

(51) Int. Cl. C12Q 1/00; G01N 27/327; G01N 27/416; G01N 27/49

(52) U.S. Cl. CPC C12Q 1/001; G01N 27/49; G01N 27/327; G01N 27/4163

(57) ABSTRACT

The internally calibrated electrochemical continuous enzyme assay (ICECEA) was developed for the fast determination of enzyme activity unit. The assay uses integration of enzyme-free pre-assay calibration with the actual enzyme assay in one continuous experiment. Such integration results in a uniquely shaped amperometric trace that allows for the selective and sensitive determination of enzymes.

### BACKGROUND OF THE INVENTION

1. Field of the Invention

The invention generally relates to methods of assessing enzyme activity. More particularly, the invention relates to the use of electrochemical assays to quantify enzyme activity.

2. Description of the Relevant Art

Enzymes are biological catalysts of great scientific and economic importance. They are one of the best-established products in biotechnology with sales of enzymes representing a billion-dollar market. Therefore, there is a high demand for simple, reliable, and cost-effective assays for the rapid evaluation of the catalytic activity of enzymes.

The majority of existing enzyme assays rely on changes in the optical properties of an enzyme solution. Frequently, such changes are due to the oxidation of dyes by the hydrogen peroxide that is produced by oxidase enzymes, or the reduction of cofactor β-nicotinamide adenine dinucleotide (NAD⁺, or NADP⁺) by dehydrogenase enzymes. However, such assays often require auxiliary enzymes and/or toxic chromogenic agents, involve a large number of liquid-handling steps, require a time-consuming incubation, and have a limited utility in turbid solutions.

Enzyme assays can also be performed by monitoring changes in the electrochemical properties of enzyme solution. Such changes are typically due to the formation or consumption of redox active species in the course of enzymatic reaction. The existing electrochemical assays have their problems too. Their selectivity can be compromised in the presence of redox active interfering species. They need extra enzyme, which can be expensive, to calibrate the measurements. They are sensitive to the activity of electrode surface, which can change upon transferring the working electrode from an assay solution to a calibration solution. This limits the precision and accuracy of unit determination.

There is, therefore, a need for improved methods of assessing and quantifying the activity of an enzyme.

### SUMMARY OF THE INVENTION

The problems discussed above may be solved by use of an internally calibrated electrochemical continuous enzyme assay (ICECEA). The ICECEA requires only a small amount of enzyme to quickly determine its activity with no need for enzyme-based external calibration or re-activation of electrode surface.

In an embodiment, the ICECEA method includes: placing a first composition in an electrochemical assay system, wherein the first composition comprises a substrate of an enzyme in a background electrolyte; adding a second composition to the first composition in the electrochemical assay system to create a first assay mixture, wherein the second composition comprises a reactant or product of an enzymatic reaction of the enzyme; measuring a current flowing through an electrode of the electrochemical assay system after the first assay mixture is formed; adding a third composition to the first assay mixture to create a second assay mixture, the third composition comprising the enzyme; measuring a current flowing through an electrode of the electrochemical assay system after the second assay mixture is formed over a predetermined time period; and determining the enzyme activity based on the change in current over time caused by the addition of the third composition. The background electrolyte may be a buffer solution (e.g., a phosphate buffer solution). The above describes a preferable way to conduct the ICECEA; however, in the alternative approach, the sequence of additions of a second composition and a third composition can be swapped.

The electrochemical assay system includes an electrochemical measuring device. The electrochemical measuring device includes working electrode, a reference electrode, and an auxiliary electrode, wherein the current flowing through the working electrode is measured. The working electrode may be a noble metal electrode, metal oxide electrode, an electrode made of a carbon allotrope, or a modified electrode. The auxiliary electrode may be a platinum wire. The reference electrode may be a Ag/AgCl/NaCl or any other reference electrode. The electrochemical assay system can also be made of only a working electrode and a reference electrode. Measuring the changes in current may be done by collecting an amperometric trace of the current.

Adding the second composition to the first composition in the electrochemical assay system includes: adding a first aliquot of the second composition to the first composition; measuring a current flowing through an electrode of the electrochemical assay system after the first aliquot is added; adding one or more additional aliquots of the second composition to the first aliquot/first composition mixture; measuring a current flowing through an electrode of the electrochemical assay system after each additional aliquot is added. Preferably, at least three aliquots of the second composition are added to the first composition before the enzyme (the third composition) is added to the mixture. Alternatively, the aliquots of the second composition are added to the first composition after the enzyme (the third composition) is added to the mixture. The enzymatic activity of the enzyme may be determined from the slope of a line created from measuring the current flowing through a working electrode of the electrochemical assay system after the second assay mixture is formed at predetermined intervals over a predetermined time period. An advantage of this method is that the addition of the second composition to the first composition and the addition of the third composition to the first assay mixture are performed in the same container using the same electrode.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 depicts a schematic amperometric trace for the ICECEA;

FIG. 2 depicts an amperometric trace for an ICECEA determination of the activity of alcohol dehydrogenase;

FIG. 3 depicts an amperometric trace for an ICECEA determination of the activity of lactate dehydrogenase;

FIG. 4 depicts an amperometric trace for an ICECEA determination of the activity of xantine oxidase;

FIG. 5 depicts an amperometric trace for an ICECEA determination of the activity of glucose oxidase;

FIG. 6A depicts an amperometric trace for a study of the effect of ascorbic acid on ICECEA quantification;

FIG. 6B depicts amperometric trace for a study of the effect of acetaminophen on ICECEA quantification;

FIG. 7 depicts an amperometric trace for an ICECEA determination of the activity of glutamate dehydrogenase;

FIG. 8 depicts an amperometric trace for an ICECEA determination of the activity of choline oxidase;

FIG. 9 depicts an amperometric trace for an ICECEA determination of the activity of glucose dehydrogenase;

FIG. 10 depicts an amperometric trace for an ICECEA determination of the activity of malic dehydrogenase;

FIGS. 11A and 11B depict an amperometric trace for an ICECEA determination of the activity of myeloperoxidase;

FIG. 12 depicts an amperometric trace for an ICECEA determination of the activity of glutamic oxaloacetate transaminase in a coupled test with malic dehydrogenase;

FIGS. 13A and B depict an amperometric trace for an ICECEA determination of the activity of alanine transaminase in a coupled test with lactate dehydrogenase;

FIG. 14 depicts an amperometric trace for an ICECEA determination of the activity of pyruvate kinase in a coupled test with lactate dehydrogenase;

FIG. 15 depicts an amperometric trace for an ICECEA determination of the activity of acetylcholinesterase in a coupled test with choline oxidase;

FIG. 16 depicts an alternate amperometric trace for an ICECEA determination of the activity of acetylcholinesterase in a coupled test with choline oxidase;

FIG. 17 depicts an alternate amperometric trace for an ICECEA determination of the activity of alanine transaminase in a coupled test with lactate dehydrogenase;

FIG. 18A depicts a schematic diagram of an electrochemical assay system;

FIG. 18B depicts a representative ICECEA trace of a product based electrochemical assay; and

FIG. 18C depicts a representative ICECEA trace of a substrate based electrochemical assay.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

It is to be understood the present invention is not limited to particular devices or methods, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Furthermore, the word “may” is used throughout this application in a permissive sense (i.e., having the potential to, being able to), not in a mandatory sense (i.e., must). The term “include,” and derivations thereof, mean “including, but not limited to.” The term “coupled” means directly or indirectly connected.

Internally Calibrated Electrochemical Continuous Enzyme Assay (ICECEA), allows for the fast determination of absolute units of enzyme activity. By definition, the enzyme activity is expressed as the amount of enzyme substrate that is converted to a product per unit time. For practical reasons, the enzyme activity is reported in the international units (1 U=1 micromole min⁻¹). The determination of enzyme activity units is based on measuring either the formation of a product or consumption of a reactant of enzymatic reaction (1) over time.

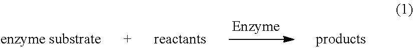

The ICECEA method measures the international activity units through the direct amperometric determination of an initial rate of reaction (1) in a single short experiment. This is feasible because of the unique integration of enzyme-less assay calibration with actual enzyme assay in one continuous experiment using the same electrode and solution. The unique feature of the new approach is that a reactant or a product of reaction (1), instead of an enzyme, is used to calibrate the assay. Calibration done in such a way does not interfere with the following enzyme assay and both can be performed in the same solution. This eliminates the need for transferring the working electrode between the calibration and assay solutions, which can affect the activity of electrode surface and lead to erroneous values for activity units. The ICECEA also eliminates the need for using enzymes, which are sometimes fairly expensive, to calibrate the assays.

The validity of enzyme assays was assured by (a) measuring the amperometric slope (initial reaction rate) only at a short reaction time, <60 s, (b) conducting an enzymatic reaction under the condition of zero order with respect to the enzyme's substrate, (c) using optimized pH and temperature, and (d) working within the linear range of a calibration plot for amperometrically monitored species. The accuracy of ICECEA was determined by calculating the relative error based on the known number of enzyme units that were added to a solution and enzyme units that were actually measured.

The ICECEA requires only three solutions to quantify the enzyme activity in the reaction 1:
A. solution of enzyme substrate and other necessary reactants in a background electrolyte;
B. solution of redox active component of enzymatic reaction;
C. solution of assayed enzyme.

The amperometric measurement is done by using any electrochemical measurement device with amperometric method and a conventional electrochemical cell with the working, reference, and counter electrodes immersed in a solution A. The working electrode is held at a potential E vs. the potential of the reference electrode. The potential E is adequate for either the oxidation or reduction of species present in a solution B. The experiment is performed by spiking one or more known aliquots of a solution B followed by one aliquot of a solution C into a stirred solution A and measuring the current flowing through the working electrode.

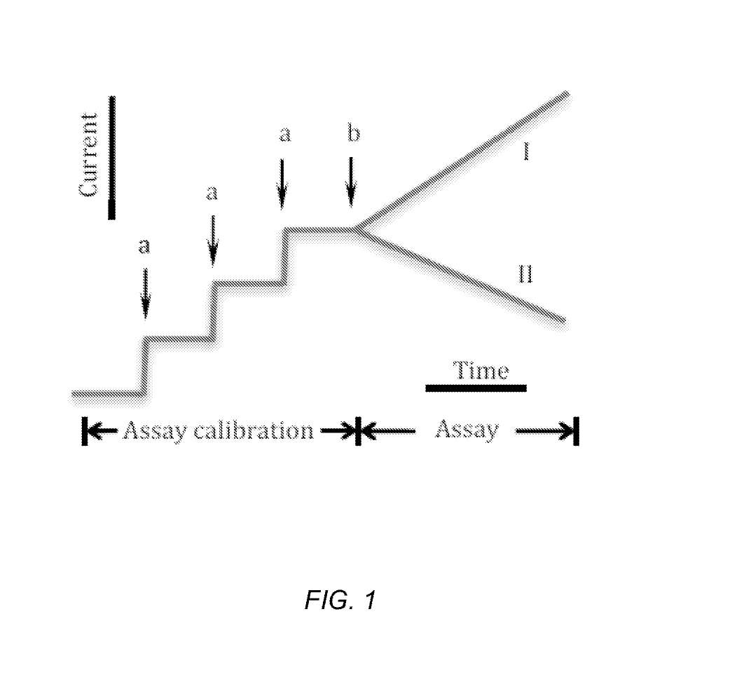

FIG. 1 shows a schematic amperometric trace, which is characteristic for the ICECEA. The trace is composed of two parts. The first part (internal pre-assay calibration) is made of three current steps that are due to the oxidation or reduction of species added with known aliquots of solution B. The arrows (a) indicate the moment of spiking the solution A with known aliquots of solution B. These current steps are used to calculate the calibration slope (CS). The second part of the amperometric trace is made of either an ascending (I) or descending (II) segment, which is recorded after the assayed enzyme is added to the solution (FIG. 1, arrow b). The segments (I) and (II) are due to the oxidation (or reduction) of a product or reactant, which are generated or consumed, respectively, by the enzymatic reaction (1). The slope of segments I and II reflects the initial rate of reaction (1) and is used to calculate the assay slope (AS). The calibration slope CS and assay slope AS are used to calculate the activity unit of an enzyme (U L⁻¹=μM min⁻¹) according to the equation

$$Units Measured ( µM {min}^{- 1} ) = \frac{AS ( µA {s}^{- 1} ) * 60 ( s {min}^{- 1} )}{CS ( µA {µM}^{- 1} )} \qquad ( 2 )$$

The ICECEA method can be used to quantitatively determine the enzyme activity of any enzymatic catalyzed reaction. The ICECEA applies directly to any enzyme that involves redox active reactants or products. It can also be coupled to other assays aimed at the determination of enzymatic activity of enzymes, which do not involve redox active species. Depending on the composition of solution B, the working electrode can be made of any electron-conducting carbon allotrope (e.g. glassy carbon, carbon nanotubes, graphite etc.) or noble metal (e.g. platinum, gold). In addition, the working electrode with a modified surface can be used to facilitate the oxidation or reduction of species added with a solution B. The time-dependent shape of the amperometric trace (FIG. 1) provides a degree of selectivity to the ICECEA method. For example, it is possible to use it in samples that contain interfering redox active species as long as these species do not act as substrates for the assayed enzyme and their concentration stays constant during the recording of the amperometric trace.

Specific applications of the ICECEA method include:
1. Determining the activity of commercial batches of enzymes
2. Measuring the levels of enzymes as biomarkers for various diseases
3. Identifying active enzymes in pools of microorganisms and enzyme mutant libraries
4. Optimizing the quantitative assays for enzymes, known or newly discovered
5. Investigating the effects of chemical environment on enzyme activity
6. Quantifying the enzyme retention in enzyme-based devices e.g. biosensors and biofuel cells
7. Corroborating high-throughput enzyme assays
8. Developing quantitative enzyme inhibition assays

The ICECEA can be performed with the variety of electrodes (e.g. platinum, glassy carbon, carbon nanotubes, modified electrodes). The method can use different redox species (e.g. H₂O₂, uric acid, NADH) to monitor the progress of enzymatic reaction by following their generation or consumption in time. The ICECEA method can be applied to wide range of enzymatic systems by matching the electrode material with the redox properties of the reactants or products of enzymatic reactions. As the field of enzyme assays is progressing rapidly, the ICECEA has a potential to play an important role in its growth.

### Examples of Redox Enzymes Amenable to Direct ICECEA Analysis

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

For the proof-of-concept purposes, the direct ICECEA was studied with several redox enzymes including the alcohol dehydrogenase (ADH), lactate dehydrogenase (LDH), xanthine oxidase (XOx), glucose oxidase (GOx), glutamate dehydrogenase (GmDH), choline oxidase (ChOx), glucose dehydrogenase (GDH), malic dehydrogenase (MDH), and myeloperoxidase (MPOx. Their determination is important for a variety of reasons. The ADH is used as a sensitive biomarker of graft function after liver transplantation. The alleviated levels of LDH are linked to the myocardial infarction, meningitis, encephalitis, and acute pancreatitis. The XOx is a biomarker of oxidative stress and plays a role in tissue and vascular injuries including liver damage, inflammatory diseases, and chronic heart failure. Finally, the GOx is commonly used as a medical diagnostic reagent and component of glucose sensors for monitoring diabetes and fermentation processes. It is also used as a food preservative. These enzymes allowed testing the ICECEA at different electrodes (carbon nanotubes, glassy carbon, and platinum) by monitoring the enzyme kinetics via the electro-oxidation of different enzyme-related species (NADH, uric acid, and hydrogen peroxide). The selected enzymes also allowed researching different pre-assay calibration strategies using either the products or reactants of enzymatic reactions. The ICECEA was conducted under the same conditions of pH and temperature as those used in the standard optical assays of these enzymes in order to assess the merits of new method.

### ICECEA of Alcohol Dehydrogenase

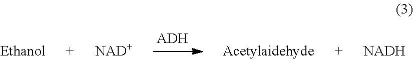

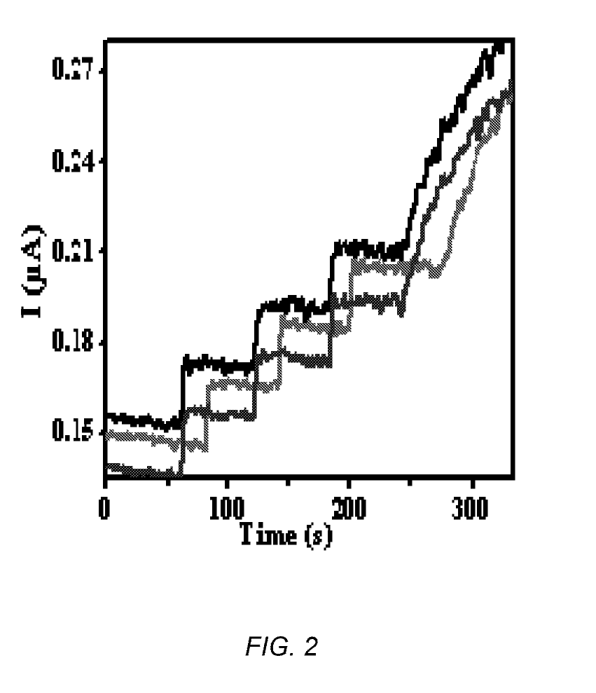

The activity of ADH was determined by measuring the initial rate of reaction (3) using three solutions: (A) 20.0 mL of 0.56 M ethanol and 7.50 mM NAD⁺ in pH 8.80 pyrophosphate buffer solution (0.050 M), (B) 1.0 mM NADH solution, (C) ADH solution (7.50 U mL⁻¹). The ICECEA was performed at 25° C. with a glassy carbon electrode (E=0.40 V) that was coated by the thin film of carbon nanotubes dispersed in chitosan, which served as an inert immobilization matrix. The CNT were used because they facilitate the oxidation of NADH. In a typical experiment, after recording a baseline current, three 20-μL aliquots of solution B and one 10-μL aliquot of solution C were added in succession to the 20.0 mL of stirred solution A. This generated characteristic ICECEA amperometric traces (FIG. 2). Calibration current steps are due to the additions of 1.0 μM NADH aliquots. The angled I-t segment was recorded after the addition of 3.74 U L⁻¹ alcohol dehydrogenase.

The current steps in FIG. 2 are due to the oxidation of NADH present in the solution B that was added in the pre-assay calibration phase of the experiment. The subsequent assay phase features an ascending I-t line, which is due to the oxidation of NADH that is generated in the enzymatic reaction (3). The latter was triggered by the addition of enzyme ADH (solution C) to the solution. The height of current steps and the initial angle of ascending line were used to determine the calibration slope CS and assay slope AS, respectively. The activity unit of ADH was calculated by using equation (2). Table 1 presents the results of such calculations based on the amperometric traces shown in FIG. 2. They demonstrate a good precision of the determination of ADH activity unit (RSD, 2.1%, N=3) based on the oxidation of NADH (generated in reaction 3) at carbon nanotubes.

TABLE 1
ICECEA of 3.74 U L⁻¹ Alcohol Dehydrogenase (pH 8.80; 25° C.)

| | | | Units Measured | Relative |
|---|---|---|---|---|
| Run # | CS (μA μM⁻¹) | AS (μA s⁻¹) | (U L⁻¹) | Error |
| 1 | 0.0200 | 1.19E−03 | 3.57 | −4.5% |
| 2 | 0.0201 | 1.20E−03 | 3.58 | −4.3% |
| 3 | 0.0204 | 1.26E−03 | 3.71 | −0.80% |
| Avg | 0.0202 | 1.22E−03 | 3.62 | −3.2% |
| RSD | 1.0% | 3.1% | 2.1% | |

Table 1 also shows a small relative error (−3.2%) between the average activity unit measured in three runs (3.62 U L⁻¹) and the unit added (3.74 U L⁻¹), which demonstrates a good accuracy of the ICECEA. Apparently, the addition of a small amount of NADH (3 μM total) to the solution during the pre-assay calibration phase did not affect notably the rate of the enzymatic reaction (3). This indicates that, under such conditions, both the pre-assay calibration with NADH and assaying of ADH can be conducted in succession in one solution and at the same electrode.

An optical assay was conducted following the procedures described in a commercial enzyme kits for alcohol dehydrogenase. The optical assay of alcohol dehydrogenase (Table 2) was much more elaborate than the ICECEA and required seven different solutions and multiple liquid-handling steps.

TABLE 2
Optical Assay of 25.0 U L⁻¹ Alcohol Dehydrogenase (pH 8.80; 25° C.)

| | | Units Measured | Relative |
|---|---|---|---|
| Run # | ΔAbs (AU min⁻¹) | (U L⁻¹) | Error |
| 1 | 0.156 | 25.0 | 0.00% |
| 2 | 0.155 | 24.9 | −0.40% |
| 3 | 0.154 | 24.8 | −0.80% |
| Avg | 0.155 | 24.9 | −0.40% |
| RSD | 0.36% | 0.36% | |

### ICECEA of Lactate Dehydrogenase

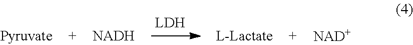

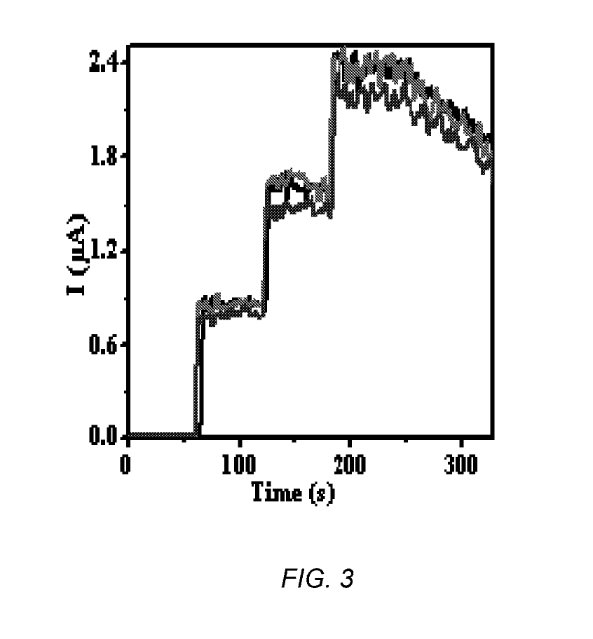

The assay of LDH was based on the reaction (4) and utilized three solutions: (A) 20.0 mL of 2.30 mM sodium pyruvate in pH 7.50 phosphate buffer solution (0.10 M), (B) 50.0 mM NADH solution, and (C) LDH solution (20.0 U mL⁻¹). The assay was performed at 37° C. with a glassy carbon electrode (E=0.40 V) that was modified by a thin film of carbon nanotubes in chitosan. The stirred solution A was spiked with three 10-μL aliquots of solution B and one 10-μL aliquot of solution C. FIG. 3 shows three representative ICECEA amperometric traces for the determination of LDH activity. Calibration current steps are due to the additions of 25.0 μM NADH aliquots. The angled descending I-t segment was recorded after the addition of 10.0 U L⁻¹ lactate dehydrogenase.

The current steps in FIG. 3 are due to the oxidation of NADH, which was added to the solution in the pre-assay calibration phase of the experiment. The subsequent assay phase features a descending I-t line that is due to the oxidation of NADH, which is consumed in the enzymatic reaction (4). The latter was triggered by the addition of LDH to the solution. The current steps and the descending slope were used to determine the calibration slope CS and assay slope AS, respectively, and calculate the activity unit of LDH using the equation (2). Table 3 shows a good precision (RSD, 2.4%, N=3) and good accuracy (relative error, 2.2%) of the determination of LDH activity unit by ICECEA. This demonstrates that the pre-assay calibration and assaying of LDH do not interfere with each other and can be conducted in the same solution and at the same electrode.

TABLE 3
ICECEA of 10.0 U L⁻¹ Lactate Dehydrogenase (pH 7.50; 37° C.)

| | | | Units Measured | Relative |
|---|---|---|---|---|
| Run # | CS (μA μM⁻¹) | AS (μA s⁻¹) | (U L⁻¹) | Error |
| 1 | 0.0320 | 5.49E−03 | 10.3 | 3.2% |
| 2 | 0.0320 | 5.55E−03 | 10.4 | 4.2% |
| 3 | 0.0298 | 4.94E−03 | 9.95 | −0.30% |
| Avg | 0.0313 | 5.33E−03 | 10.2 | 2.2% |
| RSD | 4.1% | 6.3% | 2.4% | |

The important distinction from the earlier case of ADH is that in the LDH case the pre-assay calibration plays a dual role. It provides the NADH-based assay calibration and introduces a necessary reactant (NADH) for the subsequent enzyme assay. Therefore, the calibration is done with a higher concentration of NADH (75 μM total). This assures that there is enough reactant for a reaction (4) and facilitates the recording of NADH consumption rate (descending I-t segment in FIG. 3) in order to determine the LDH activity. The good precision and accuracy of this approach demonstrates the flexibility of the ICECEA concept.

An optical assay was conducted following the procedures described in a commercial enzyme kits for lactate dehydrogenase. The optical assay of lactate dehydrogenase (Table 4) was more elaborate and required five different solutions and much more time compared to the ICECEA.

TABLE 4
Optical Assay of 16.7 U L⁻¹ Lactate Dehydrogenase (pH 7.50; 37° C.)

| | ΔAbs | Units Measured | Relative |
|---|---|---|---|
| Run # | (AU min⁻¹) | (U L⁻¹) | Error |
| 1 | 0.105 | 16.9 | 1.2% |
| 2 | 0.107 | 17.2 | 3.0% |
| 3 | 0.103 | 16.5 | −1.2% |
| Avg | 0.105 | 16.9 | 1.2% |
| RSD | 2.1% | 2.1% | |

### ICECEA of Xantine Oxidase

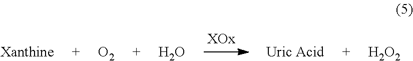

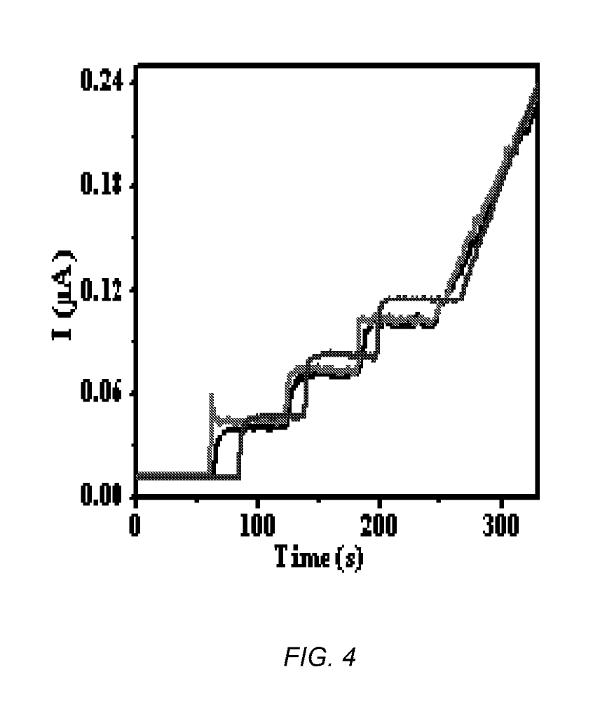

The activity of XOx was determined by measuring the initial rate of reaction (5) using three solutions: (A) 20.0 mL of 0.50 mM xanthine in pH 7.50 phosphate buffer solution (0.10 M), (B) 2.50 mM solution of uric acid, and (C) XOx solution (8.06 U mL⁻¹). The enzyme XOx is different from the previous two enzymes because its reaction involves three species (O₂, uric acid, and H₂O₂) that can be reduced or oxidized at conventional electrodes. In principle, any of them could be used to assay the XOx. In order to simplify the measurement and monitor only one type of species, a glassy carbon electrode was selected to monitor the oxidation of uric acid that is produced in reaction (5). Out of the three species, uric acid is the only species that is oxidized at such electrode at 0.35 V. The assay was performed at 25° C. The stirred solution A was spiked with three 20-μL aliquots of solution B and one 20-μL aliquot of solution C. FIG. 4 shows the representative ICECEA amperometric traces for the XOx assay. Calibration current steps are due to the additions of 2.5-μM uric acid aliquots. The angled I-t segment was recorded after the addition of 8.03 U L⁻¹ xanthine oxidase.

The current steps in FIG. 4 are due to the oxidation of aliquots of uric acid (solution B) that were added to the solution A in the pre-assay calibration phase of the measurement. The following ascending I-t line is due to the oxidation of uric acid, which is generated by the enzymatic reaction 5 that was triggered by the addition of XOx to the solution. The current steps and the ascending I-t line were used to determine the calibration slope CS and assay slope AS, respectively, and calculate the activity unit of XOx according to equation 2. Table 5 shows the results for the three typical runs presented in FIG. 4. They demonstrate a good precision (RSD, 2.8%, N=3) and accuracy (relative error, 0.12%) of the determination of XOx activity unit by the ICECEA.

The good precision and accuracy was achieved regardless of much larger standard deviation for the slopes CS and AS (8.6% and 11%, respectively). Apparently, the differences in slopes between different runs did not impact the quality of unit determination. The reason for this was that the activity unit was calculated based on an experiment that did not require transferring the working electrode between the separate calibration and assay solutions. This provided internal consistency between the calibration and assay phases of an individual run. This is an inherent advantage of ICECEA method, which provides reliable results without the need for re-polishing of working electrode.

TABLE 5
ICECEA of 8.03 U L⁻¹ Xanthine Oxidase (pH 7.50; 25° C.)

| | | | Units Measured | Relative |
|---|---|---|---|---|
| Run # | CS (μA μM⁻¹) | AS (μA s⁻¹) | (U L⁻¹) | Error |
| 1 | 0.0121 | 1.61E−03 | 7.98 | −0.62% |
| 2 | 0.0123 | 1.61E−03 | 7.85 | −2.2% |
| 3 | 0.0141 | 1.95E−03 | 8.30 | 3.4% |
| Avg | 0.0128 | 1.72E−03 | 8.04 | 0.12% |
| RSD | 8.6% | 11% | 2.8% | |

The relative error between the average activity unit measured in three runs (8.04) and the unit added (8.03) to the solution was only 0.12% (Table 5) illustrating that the pre-assay calibration with uric acid and assaying of XOx did not interfere with each other and could be conducted in the same solution.

An optical assay was conducted following the procedures described in a commercial enzyme kits for xanthine oxidase. The optical assay of xanthine oxidase (Table 6) was similar to the ICECEA in that it required the same number of solutions and comparable amount of time to determine the enzyme activity.

TABLE 6
Optical Assay of 6.67 U L⁻¹ Xanthine Oxidase (pH 7.50; 25° C.).

| | | Units Measured | Relative |
|---|---|---|---|
| Run # | ΔAbs (AU min⁻¹) | (U L⁻¹) | Error |
| 1 | 0.0837 | 6.86 | 2.8% |
| 2 | 0.0843 | 6.91 | 3.6% |
| 3 | 0.0811 | 6.65 | −0.30% |
| Avg | 0.0830 | 6.81 | 2.1% |
| RSD | 2.0% | 2.0% | |

### ICECEA of Glucose Oxidase

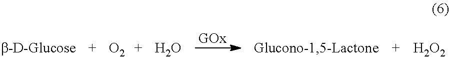

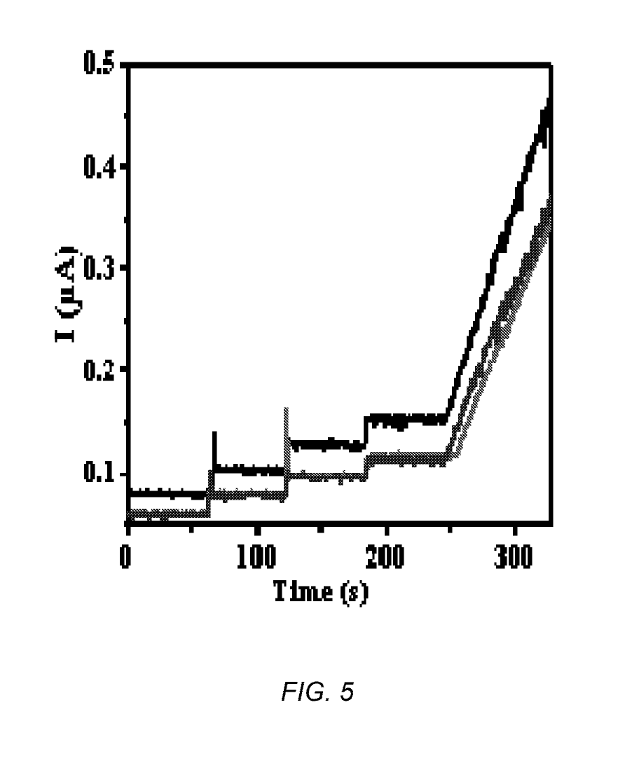

The assay of enzyme GOx was based on reaction (6) and three solutions: (A) 20.0 mL of 0.10 M β-D-Glucose in pH 5.1 acetate buffer solution (0.10 M), (B) 0.0020 M solution of H₂O₂, and (C) GOx solution (40 U mL⁻¹). The assay was performed at 35° C. by using a platinum working electrode that was held at E=0.60 V. The solution A was spiked with three 20-μL aliquots of solution B and one 10-μL aliquot of solution C. FIG. 5 shows the representative ICECEA amperometric traces for GOx assay. Calibration current steps are due to the additions of 2.0-μM H₂O₂ aliquots. The angled I-t segment was recorded after the addition of 19.9 U L⁻¹ glucose oxidase. The current steps in FIG. 5 are due to the oxidation of H₂O₂ (solution B) that was added to a stirred solution A in the pre-assay calibration phase. The subsequent assay phase features an ascending I-t line that is due to the oxidation of H₂O₂, which is generated by the enzymatic reaction 6 that was triggered by the addition of GOx to the solution.

The analysis of I-t traces (FIG. 5) is presented in Table 7, which shows a good precision (RSD, 2.7%, N=3) and good accuracy (relative error, −1.0%) of the determination of GOx activity unit by ICECEA. Again, as in the case of XOx, a good accuracy and precision was achieved regardless of the much larger RSD for the slopes CS and AS (16% and 13%, respectively).

TABLE 7
ICECEA of 19.9 U L⁻¹ Glucose Oxidase (pH 5.10; 35° C.)

| | | | Units Measured | Relative |
|---|---|---|---|---|
| Run # | CS (μA μM⁻¹) | AS (μA s⁻¹) | (U L⁻¹) | Error |
| 1 | 1.22E−02 | 3.91E−03 | 19.2 | −3.5% |
| 2 | 9.60E−03 | 3.15E−03 | 19.7 | −1.2% |
| 3 | 9.20E−03 | 3.11E−03 | 20.3 | 1.8% |
| Avg | 1.03E−02 | 3.39E−03 | 19.7 | −1.0% |
| RSD | 16% | 13% | 2.7% | |

An optical assay was conducted following the procedures described in a commercial enzyme kits for glucose oxidase. The optical assay of glucose oxidase (Table 8) was much more time consuming than the ICECEA and required an auxiliary enzyme peroxidase, toxic dye o-dianisidine, and four other solutions to determine the enzyme activity.

TABLE 8
Optical Assay of 19.4 U L⁻¹ Glucose Oxidase (pH 5.10; 35° C.)

| | ΔAbs | Units Measured | Relative |
|---|---|---|---|
| Run # | (AU min⁻¹) | (U L⁻¹) | Error |
| 1 | 0.150 | 0.0200 | 3.1% |
| 2 | 0.149 | 0.0199 | 2.6% |
| 3 | 0.152 | 0.0202 | 4.1% |
| Avg | 0.150 | 0.0200 | 3.1% |
| RSD | 0.80% | 0.80% | |

### Glutamate Dehydrogenase (GmDH)

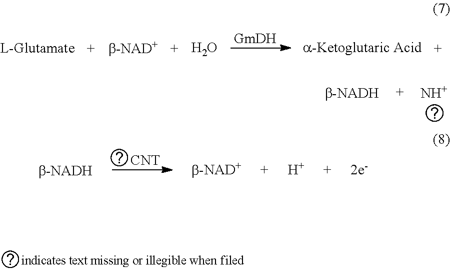

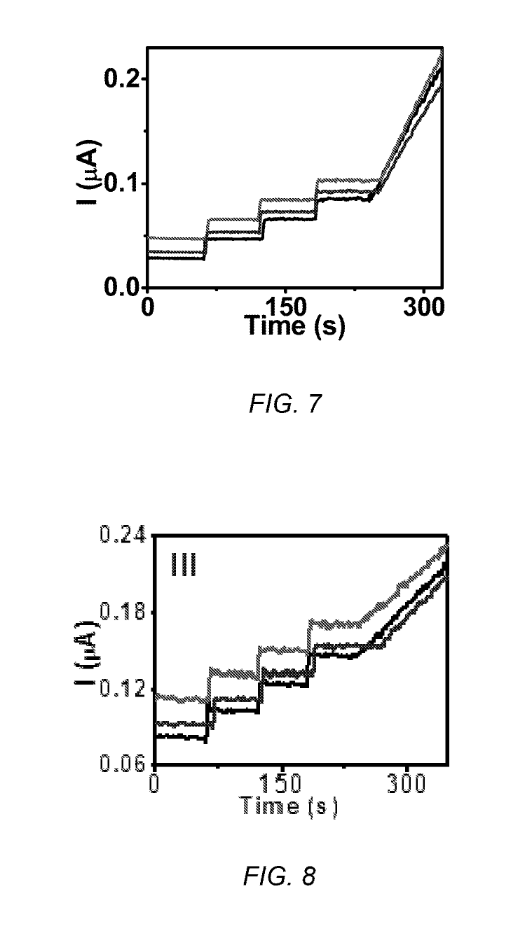

FIG. 7 depicts ICECEA current-time traces (E=0.40 V) recorded at a glassy carbon/CNT film electrode in a stirred solution of 0.15 M glutamate in 0.10 M triethanolamine buffer (Solution A, pH 7.30, 21° C.). The calibration I-t steps are due to the additions of 1.0 μM aliquots of NADH (Solution B). The angled I-t segment was recorded after the addition of 9.97 U L⁻¹ GmDH (Solution C). The current steps in FIG. 7 are due to the increase in the amount of NADH when solution B was added to a stirred solution A in the pre-assay calibration phase. The subsequent assay phase features an ascending I-t line that is due to the increase in NADH, which is generated by the enzymatic reaction 7 that was triggered by the addition of GmDH to the solution.

The analysis of I-t traces (FIG. 7) is presented in Table 9, which shows a good precision and good accuracy of the determination of GmDH activity unit by ICECEA.

TABLE 9
ICECEA of 9.97 U L⁻¹ Glutamate Dehydrogenase (pH, 7.3; 21° C.)

| | | | Units Measured | Relative |
|---|---|---|---|---|
| Run # | CS (μA μM⁻¹) | AS (μA s⁻¹) | (U L⁻¹) | Error (%) |
| 1 | 9.49E−03 | 1.62E−03 | 10.2 | 2.7 |
| 2 | 9.34E−03 | 1.62E−03 | 10.4 | 4.4 |
| 3 | 9.67E−03 | 1.56E−03 | 9.68 | −2.9 |
| Avg | 9.50E−03 | 1.60E−03 | 10.1 | 1.4 |
| RSD | 1.7% | 2.2% | 3.8% | |

### Choline Oxidase (ChOx)

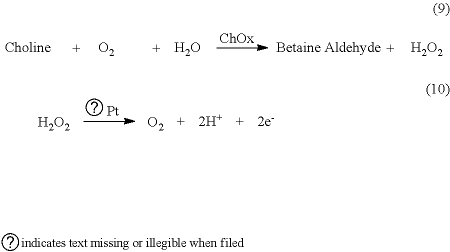

FIG. 8 depicts ICECEA amperometric traces (E=0.60 V) recorded at a platinum electrode in a stirred solution of 0.050 M choline in 0.10 M Tris buffer (Solution A, pH 8.0, 37° C.). Calibration current steps are due to the additions of 2.0 μM H₂O₂ aliquots (Solution B). The angled I-t segment was recorded after the addition of 3.74 U L⁻¹ ChOx (Solution C). The current steps in FIG. 8 are due to the increase in the amount of H₂O₂ when solution B was added to a stirred solution A in the pre-assay calibration phase. The subsequent assay phase features an ascending I-t line that is due to the increase in H₂O₂, which is generated by the enzymatic reaction 9 that was triggered by the addition of ChOx to the solution.

The analysis of I-t traces (FIG. 8) is presented in Table 10, which shows a good precision and good accuracy of the determination of ChOx activity unit by ICECEA.

TABLE 10
ICECEA of 3.74 U L⁻¹ Choline Oxidase (pH, 8.0; 37° C.)

| | | | Units Measured | Relative |
|---|---|---|---|---|
| Run # | CS (μA μM⁻¹) | AS (μA s⁻¹) | (U L⁻¹) | Error |
| 1 | 0.011 | 7.15E−04 | 3.90 | 4.3% |
| 2 | 0.010 | 6.19E−04 | 3.71 | −0.70% |
| 3 | 0.011 | 6.71E−04 | 3.66 | −2.1% |
| Avg | 0.011 | 6.68E−04 | 3.76 | 0.53% |
| RSD | 5.4% | 7.2% | 3.4% | |

### Glucose Dehydrogenase (GDH)

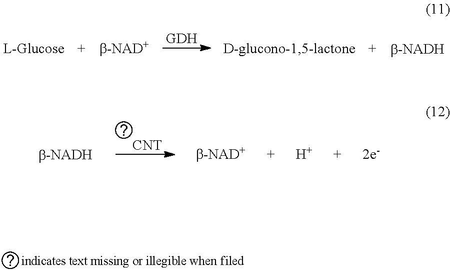

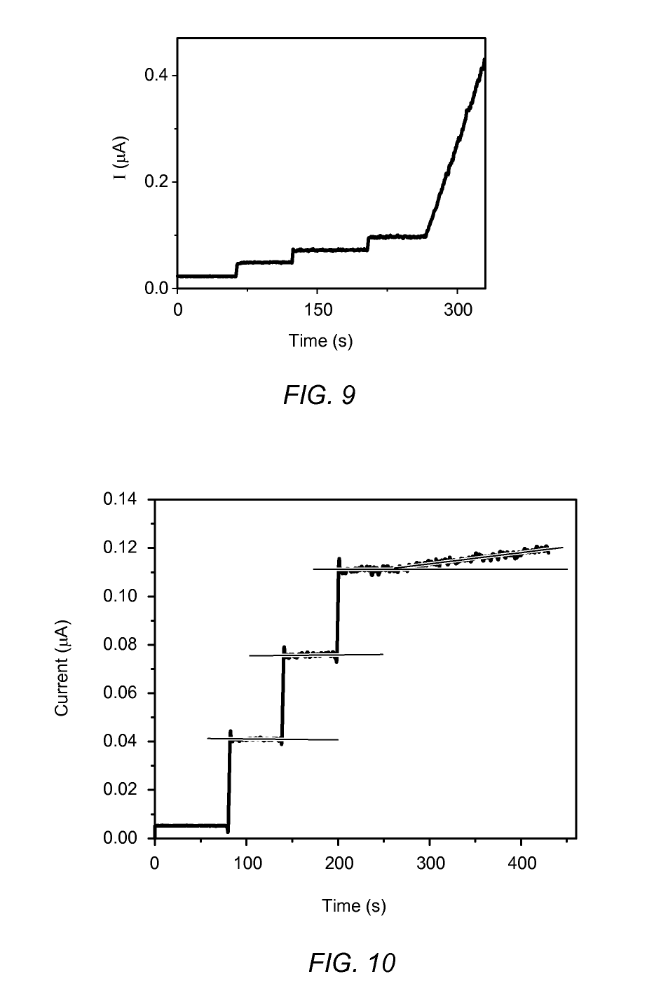

FIG. 9 depicts ICECEA current-time traces (E=0.40 V) recorded at a glassy carbon/CNT film electrode in a stirred solution of 0.50 M glucose and 4.0 mM NAD⁺ in 0.050 M phosphate buffer (Solution A, pH 7.40, 21° C.). The calibration I-t steps are due to the additions of 10.0 μM aliquots of NADH (Solution B). The angled I-t segment was recorded after the addition of 8.87 U L⁻¹ glucose dehydrogenase (Solution C). The current steps in FIG. 9 are due to the increase in the amount of NADH when solution B was added to a stirred solution A in the pre-assay calibration phase. The subsequent assay phase features an ascending I-t line that is due to the increase in NADH, which is generated by the reaction 11 that was triggered by the addition of GDH to the solution.

### Malic Dehydrogenase (MDH)

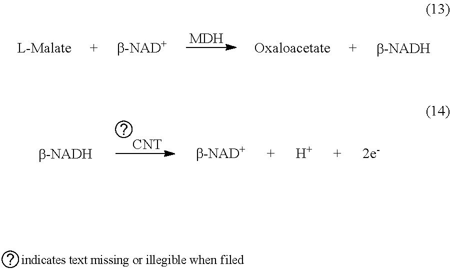

FIG. 10 depicts ICECEA current-time traces (E=0.40 V) recorded at a glassy carbon/CNT film electrode in a stirred solution of 4.0 mM malic acid in 0.050 M phosphate (K+) buffer (Solution A, pH 7.60, 21° C.). The calibration I-t steps are due to the additions of 2.0 μM aliquots of NADH (Solution B). The angled I-t segment was recorded after the addition of 10.0 U L⁻¹ MDH (Solution C). The current steps in FIG. 10 are due to the increase in the amount of NADH when solution B was added to a stirred solution A in the pre-assay calibration phase. The subsequent assay phase features an ascending I-t line that is due to the increase in NADH, which is generated by the reaction 13 that was triggered by the addition of MDH to the solution.

### Myeloperoxidase (MPOx)

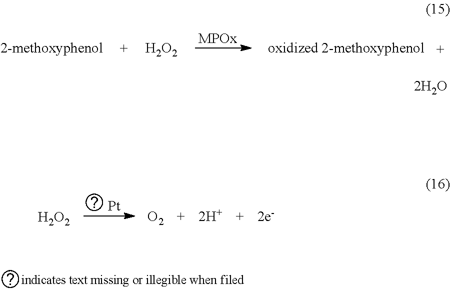

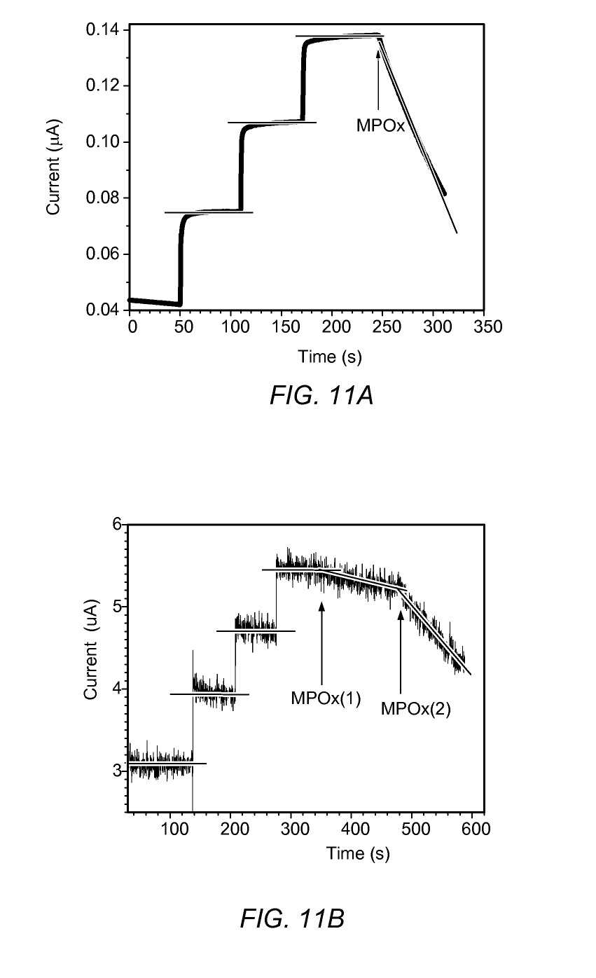

FIG. 11A depicts ICECEA current-time traces (E=0.60 V) recorded at a Pt electrode in a stirred solution of 100.0 mM 2-methoxyphenol in 0.050 M phosphate (K+) buffer (Solution A, pH 7.00, 21° C.). The calibration I-t steps are due to the additions of 0.200 mM aliquots of H₂O₂ (Solution B). The angled I-t segment was recorded after the addition of 50.0 U L⁻¹ MPOx (Solution C). The current steps in FIG. 11A are due to the increase in the amount of H₂O₂ when solution B was added to a stirred solution A in the pre-assay calibration phase. The subsequent assay phase features a descending I-t line that is due to the decrease in H₂O₂, which is consumed by the reaction 15 that was triggered by the addition of MPOx to the solution.

FIG. 11B depicts ICECEA current-time traces recorded via differential pulse amperometry (DPA) at a Pt electrode in a stirred solution of 100.0 mM 2-methoxyphenol in 0.050 M phosphate (K+) buffer (Solution A, pH 7.00, 21° C.). DPA parameters: cleaning E—0.300 V for 100 ms; pulse E₁—0.100 V for 100 ms; pulse E₂—0.300 V for 100 ms. The calibration I-t steps are due to the additions of 0.200 mM aliquots of H₂O₂ (Solution B). The angled I-t segments were recorded after the addition of 4.9 and 12.2 U L⁻¹ MPOx (Solution C). The current steps in FIG. 11B are due to the increase in the amount of H₂O₂ when solution B was added to a stirred solution A in the pre-assay calibration phase. The subsequent assay phase features a descending I-t line that is due to the decrease in H₂O₂, which is consumed by the reaction 15 that was triggered by the addition of MPOx to the solution.

In the alternative approach to the determination of MPOx, the reaction with thiocyanate was used.

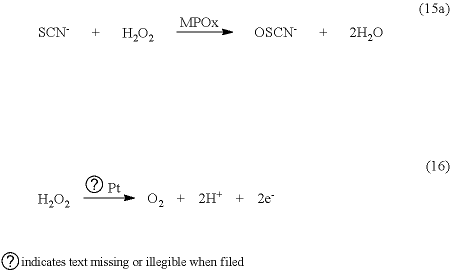

The ICECEA based on reactions 15a and 16 yielded the same current-time traces as those shown in FIG. 11A. They were recorded at a Pt electrode (E=0.40 V) in a stirred solution of 10.0 mM NH₄SCN in 0.050 M phosphate (K+) buffer (Solution A, pH 7.00, 21° C.). The calibration I-t steps were due to the additions of 20.0 μM aliquots of H₂O₂ (Solution B). The angled I-t segment was recorded after the addition of 5.0 U L⁻¹ MPOx (Solution C).

### Interference Studies

The initial interference studies were performed with the enzyme GOx and ascorbic acid. The latter is notorious for interfering with electrochemical assays because it can be easily oxidized at working electrodes. The addition of physiological concentration (0.10 mM) of ascorbic acid to the enzyme solution C did not change the ICECEA traces (not shown). This indicated that the ascorbic acid did not interfere with the electrochemical assaying of GOx under such conditions. Apparently, the high dilution factor

$$( 2000 = \frac{20.01 mL of solution A}{0.010 mL of solution C} )$$

of the solution C during the assay lowered the concentration of ascorbic acid to a level (50 nM) that was not detectable at a platinum electrode.

The situation changed when the dilution factor was decreased from 2000 to 20

$$( 20 = \frac{19.0 mL of solution A + 1.0 mL of solution C}{1.0 mL of solution C} )$$

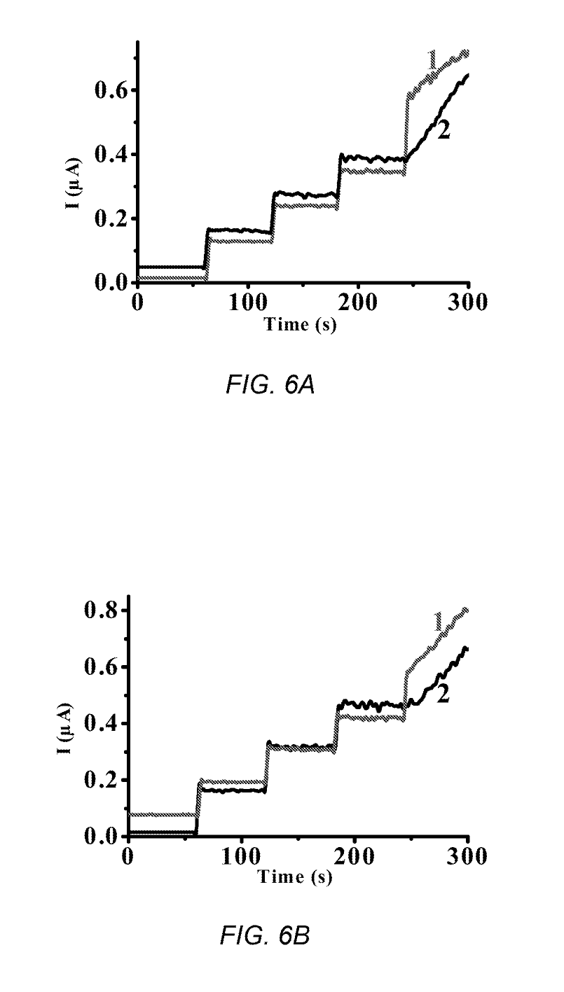

resulting in a 15 μM ascorbic acid solution. In the presence of 15 μM ascorbic acid in the assay mixture (FIG. 6A, trace 1), the angled I-t segment of ICECEA trace at >250 s shifted upward when compared to that recorded without the ascorbic acid (FIG. 6A, trace 2). The shift was due to the extra current that was generated by the oxidation of ascorbic acid at the surface of platinum electrode. The second significant change was a decrease in the slope of angled I-t segment in the presence of ascorbic acid in the solution by 50%. This indicated that the ascorbic acid at higher concentration interfered with the measurement of enzyme activity. The interference could be ascribed to the reaction of ascorbic acid, which is a relatively strong reducing agent, with hydrogen peroxide that was generated in the enzymatic reaction. As hydrogen peroxide was consumed by ascorbic acid, less hydrogen peroxide was available for the oxidation at the platinum electrode. Therefore, less current was generated at the electrode leading to a decrease in the I-t slope.

The second part of interference studies was conducted with acetaminophen, which is also known for interfering with electrochemical assays. In the presence of acetaminophen, the angled I-t segment of ICECEA trace shifted upward (FIG. 6B, trace 1) as in the case of ascorbic acid. This increase in current was due to the oxidation of acetaminophen at the platinum electrode. However, the I-t segment remained practically parallel to that recorded in the absence of acetaminophen (FIG. 6B, trace 2). The difference between the slopes was below 3%. This is significant because it indicates that the acetaminophen, which is a milder reducing agent than ascorbic acid, did not interfere with the determination of enzyme activity. Apparently, the unique shape of the ICECEA I-t trace, which is composed of rapid and more gradual changes in current, allows for the selective determination of enzyme activity even in the presence of redox active species such as acetaminophen as long as their concentration stays constant during the measurement.

The sensitivity of I-t slope to the composition of solution (FIG. 6) indicates that the ICECEA, in addition to quantify the enzyme activity, can also quickly screen for potential interfering species, enzyme inhibitors, and enzyme substrates.

### Detection Limits for Enzyme Activity Units

The capacity of ICECEA to reliably quantify enzyme activity depends on the limit of detection (LOD) for the molecules that are electrochemically monitored during the assay (NADH, uric acid, H₂O₂) and the linear range of their calibration plots. For the purpose of this analysis, the LOD is defined as the concentration that generates the current I_{LOD} that is three times larger than the peak-to-peak noise of baseline current. The low limit of enzyme detection (LLED) and high limit of enzyme detection (HLED) were determined under the condition that (1) the enzyme was always a limiting reagent, and (2) the current measured during the assay was always within the linear range of a calibration plot for the monitored species. The LLED values were calculated by using the equation 2 and AS=I_{LOD}/2 min, which yielded 0.090, 0.024, 0.0031, and 0.036 U L⁻¹ (or 2.1, 0.12, 89, and 2.0 pM) for the ADH, LDH, XOx, and GOx, respectively. These LLED values are competitive and sometimes better by orders of magnitude than those previously reported for these enzymes.

As the enzyme concentration in a solution increased the segments 1 and 2 became progressively less linear. Therefore, the HLED was determined based on the slope AS that was calculated by selecting a shorter (<30 s) linear portion of segment I, which had correlation coefficient R²>0.99 (>0.97 for segment II (FIG. 1)). Following this approach, the HLED values of 47, 55, 54, and 105 U L⁻¹ (or 1.1 nM, 0.27 nM, 1.5 μM, and 5.6 nM) were obtained for the ADH, LDH, XOx, and GOx, respectively.

### Examples of Enzymes Amenable to ICECEA Analysis via Coupled Assays

Coupled ICECEA. Many enzymatic reactions are difficult to assay directly by amperometry because their products or reactants are electrochemically inactive. Therefore, the coupled ICECEA will be used based on the set of general enzymatic reactions 17 and 18

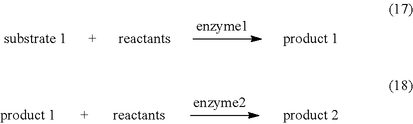

in which the rate determining reaction 17 is coupled with reaction 18 that is easier detectable at a proper working electrode. In the coupled assays, the product of reaction 17 (product 1) will be the substrate of reaction 18. The coupled ICECEA assays control the direction of enzymatic reactions in order to rapidly extract the information about the enzyme activity under linear conditions.

The reaction 18 can be also conducted in electrochemical biosensors as the alternative to conducting it in the assay solutions. To this end, the enzyme 2 is immobilized in a thin polymeric film (e.g. chitosan, Nafion etc.) on the surface of electrode made of an electronic conductor or in electronically conducting pastes. Such biosensors are convenient alternatives, lowering the cost of ICECEA analysis by requiring the use of only small quantities of expensive enzymes and allowing their multiple reuse.

### I. Glutamic Oxaloacetate Transaminase (GOT)/Malic Dehydrogenase (MDH)

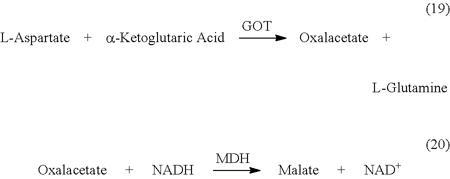

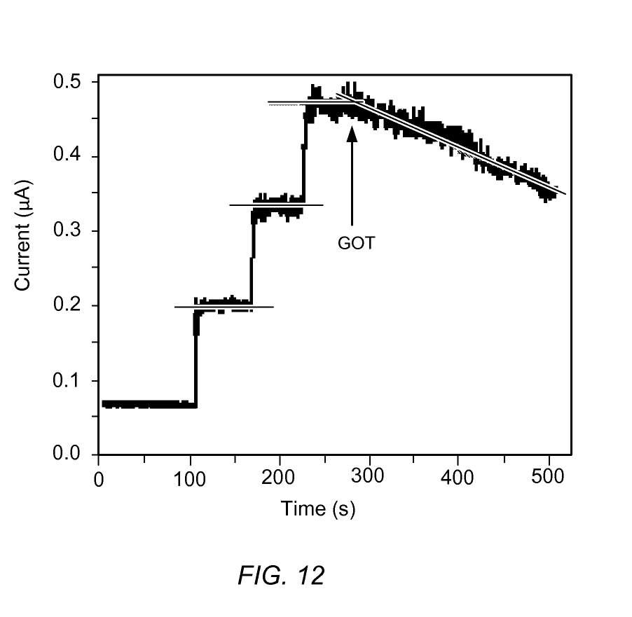

FIG. 12 depicts an ICECEA current-time traces (E=0.40 V) recorded at a glassy carbon/CNT film electrode in a stirred solution of 50.0 mM aspartate, 12.5 mM ketoglutaric acid, and 600 U L−1 MDH in 0.10 M Tris buffer (Solution A, pH 7.80, 37° C.). The calibration I-t steps are due to the additions of 5.0 μM aliquots of NADH (Solution B). The angled I-t segment was recorded after the addition of 1.0 U L⁻¹ GOT (Solution C). The current steps in FIG. 12 are due to the increase in the amount of NADH when solution B was added to a stirred solution A in the pre-assay calibration phase. The subsequent assay phase features a descending I-t line that is due to the decrease in NADH, which is consumed by the reaction 20 that was triggered by the addition of GOT to the solution. Addition of GOT catalyzes the reaction of aspartate with ketoglutaric acid to form oxaloacetate, which serves as a substrate for reaction 20. The rate of consumption of NADH is directly related to the amount of oxaloacetate produced in the GOT catalyzed reaction. Thus the change in concentration of NADH can be directly related to the activity of GOT.

### II. Alanine Transaminase (ALT)/Lactate Dehydrogenase (LDH)

### ALT is Also Called Glutamic Pyruvic Transaminase, GPT

$$\begin{matrix}\frac{L \text{-} Alanine + a \text{-} Ketoglutaric Acid {\to }^{•} ALT}{PLP} Pyrurate + L \text{-} Glutamine & ( 21 ) \\ Pyrurate + NADH {\to }^{LDH} Lactate + {NAD}^{\to } & ( 22 )\end{matrix}$$

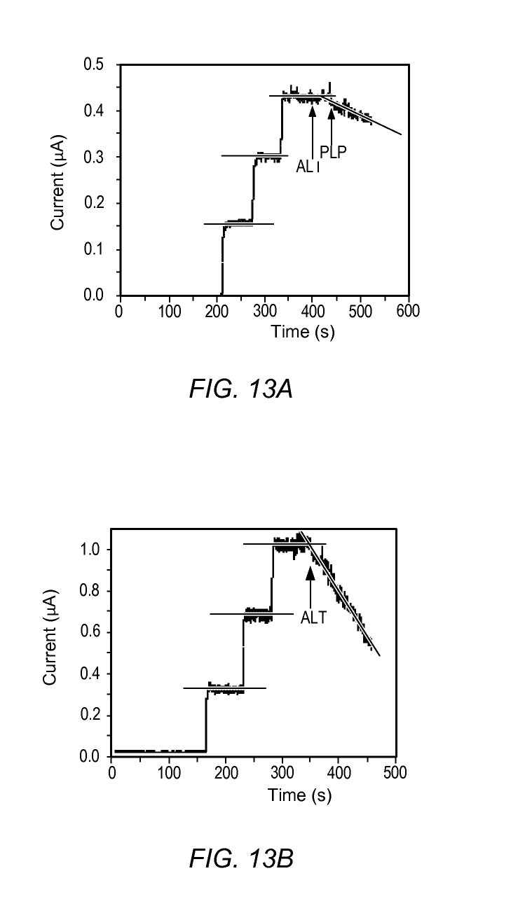

FIG. 13A depicts an ICECEA current-time trace (E=0.40 V) recorded at a glassy carbon/CNT film electrode in a stirred solution of 100.0 mM alanine, 10.0 mM ketoglutaric acid, 0.10 mM pyridoxal phosphate (PLP), and 1200 U L⁻¹ LDH in 0.10 M Tris buffer (Solution A, pH 7.40, 21° C.). The calibration I-t steps are due to the additions of 8.0 μM aliquots of NADH (Solution B). The angled I-t segment was recorded after the addition of 100.0 U L⁻¹ ALT (Solution C). The current steps in FIG. 13A are due to the increase in the amount of NADH when solution B was added to a stirred solution A in the pre-assay calibration phase. The subsequent assay phase features a descending I-t line that is due to the decrease in NADH, which is consumed by the reaction 22 that was triggered by the addition of ALT to the solution. Addition of ALT catalyzes the reaction of alanine with ketoglutaric acid to form pyruvate, which serves as a substrate for reaction 22. The rate of consumption of NADH is directly related to the amount of pyruvate produced in the ALT catalyzed reaction. Thus the change in concentration of NADH can be directly related to the activity of ALT.

FIG. 13B depicts an ICECEA current-time traces (E=0.40 V) recorded at a glassy carbon/CNT film electrode in a stirred solution of 200.0 mM alanine, 10.0 mM ketoglutaric acid, 1.0 mM pyridoxal phosphate (PLP), and 6000 U L⁻¹ LDH in 0.10 M Tris buffer (Solution A, pH 7.40, 37° C.). The calibration I-t steps are due to the additions of 40.0 μM aliquots of NADH (Solution B). The angled I-t segment was recorded after the addition of 50.0 U L⁻¹ ALT (Solution C).

### III. Alanine Transaminase (ALT)/Pyruvate Oxidase (PyOx)

$$\begin{matrix}\frac{L \text{-} Alanine + a \text{-} Ketoglutaric Acid {\to }^{•} ALT}{PLP} Pyrurate + L \text{-} Glutamine & ( 23 ) \\ Pyrurate + phosphate + {O}_{2} {\to }^{PyOx} Acetyl phosphate + {CO}_{2} + {H}_{2} {O}_{2} & ( 24 )\end{matrix}$$

This assay may be performed by treating a stirred solution of alanine, ketoglutaric acid, pyridoxal phosphate (PLP), and PyOX in an appropriate buffer (Solution A). The calibration I-t steps are performed by additions of aliquots of H₂O₂ (Solution B). The angled I-t segment is recorded after addition of 100.0 U L⁻¹ ALT (Solution C). Addition of ALT catalyzes the reaction of alanine with ketoglutaric acid to form pyruvate, which serves as a substrate for reaction 24. The rate of increase of H₂O₂ is directly related to the amount of pyruvate produced in the ALT catalyzed reaction. Thus the change in concentration of H₂O₂ can be directly related to the activity of ALT.

### IV. Pyruvate Kinase (PyK)/Lactate Dehydrogenase (LDH)

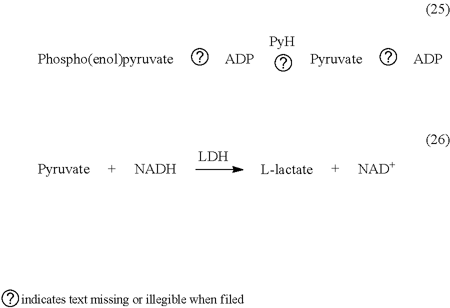

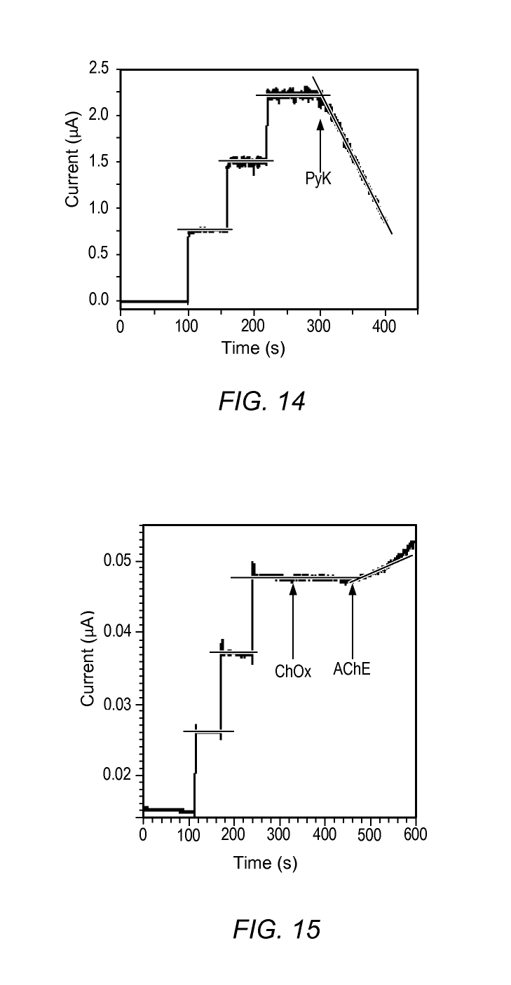

FIG. 14 depicts ICECEA current-time traces (E=0.40 V) recorded at a glassy carbon/CNT film electrode in a stirred solution of 0.60 mM phospho(enol)pyruvate (PEP), 1.50 mM adenosine 5′-diphosphate (ADP), 7.0 mM MgSO₄, and 3000 U L⁻¹ LDH in 0.050 M phosphate (K⁺) buffer (Solution A, pH 7.60, 21° C.). The calibration I-t steps are due to the additions of 40.0 μM aliquots of NADH (Solution B). The angled I-t segment was recorded after the addition of 200.0 U L⁻¹ PyK (Solution C). The current steps in FIG. 14 are due to the increase in the amount of NADH when solution B was added to a stirred solution A in the pre-assay calibration phase. The subsequent assay phase features a descending I-t line that is due to the decrease in NADH, which is consumed by the reaction 26 that was triggered by the addition of PyK to the solution. Addition of PyK catalyzes the reaction of phosphor(enol)pyruvate with ADP to form pyruvate, which serves as a substrate for reaction 26. The rate of consumption of NADH is directly related to the amount of pyruvate produced in the PyK catalyzed reaction. Thus the change in concentration of NADH can be directly related to the activity of PyK.

### V. Pyruvate Kinase (PyK)/Pyruvate Oxidase (PyOx)

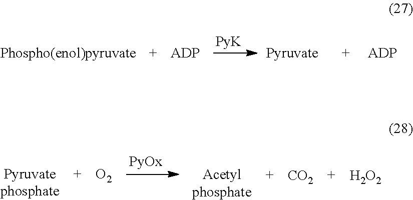

This assay may be performed by treating a stirred solution of Phospho(enol)pyruvate, ADP, MgSO₄, and PyOx in an appropriate buffer (Solution A). The calibration I-t steps are performed by additions of aliquots of H₂O₂ (Solution B). The angled I-t segment is recorded after addition of 100.0 U L⁻¹ PyK (Solution C). Addition of PyK catalyzes the reaction of phospho(enol)pyruvate with ADP to form pyruvate, which serves as a substrate for reaction 28. The rate of increase of H₂O₂ is directly related to the amount of pyruvate produced in the PyK catalyzed reaction. Thus the change in concentration of H₂O₂ can be directly related to the activity of PyK.

### IV. Acetylcholinesterase/Choline Oxidase (AChE/ChOx)

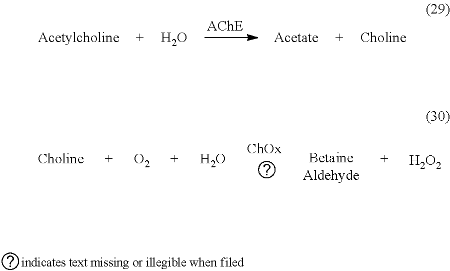 [SMILES: O ; "O2" ; O ; "H2O2" ; "BetaineAldehyde"]

FIG. 15 depicts an ICECEA current-time traces (E=0.60 V) recorded at a Pt electrode in a stirred solution of 4.0 mM acetylcholine, 14.0 mM MgSO₄, and 52 U L⁻¹ ChOx in 0.10 M Tris buffer (Solution A, pH 8.00, 21° C.). The calibration I-t steps are due to the additions of 2.0 μM aliquots of H₂O₂ (Solution B). The angled I-t segment was recorded after the addition of 50.0 U L⁻¹ AChE (Solution C). Addition of AChE catalyzes the reaction of acetylcholine with AChE to form choline, which serves as a substrate for reaction 30. The rate of increase of H₂O₂ is directly related to the amount of choline produced in the AChE catalyzed reaction. Thus the change in concentration of H₂O₂ can be directly related to the activity of AChE.

### Consecutive ICECEA Determination of Activity of Different Enzymes in the Same Solution

### Proof-of-Concept #1: Acetylcholinesterase (AChE) and Choline Oxidase (ChOx)

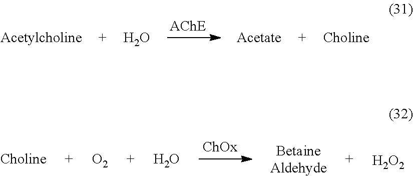 [SMILES: O ; "O2" ; O ; "BetaineAldehyde" ; "H2O2"]

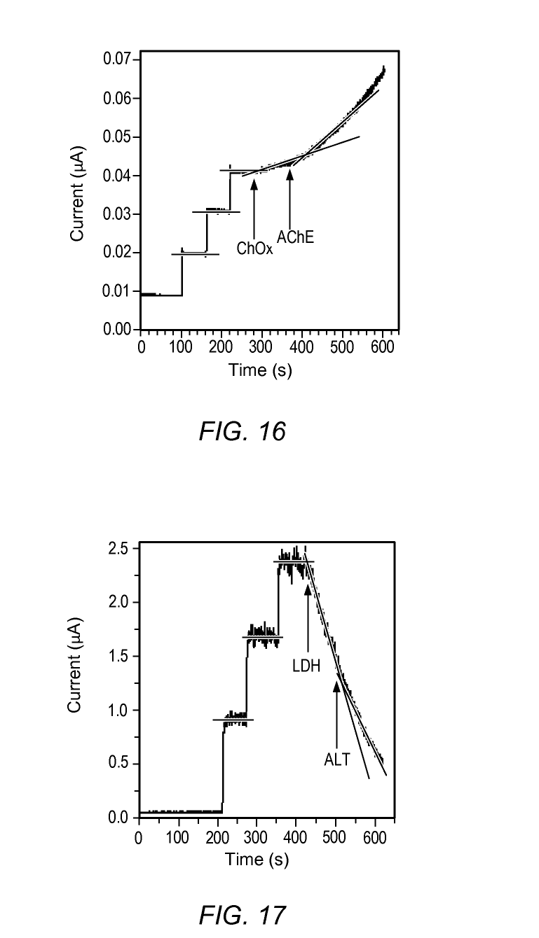

FIG. 16 depicts an ICECEA current-time traces (E=0.60 V) recorded at a Pt electrode in a stirred solution of 4.0 mM acetylcholine, 14.0 mM MgSO₄, and 1.4 μM choline in 0.10 M Tris buffer (Solution A, pH 8.00, 21° C.). The calibration I-t steps are due to the additions of 2.0 μM aliquots of H₂O₂ (Solution B). The angled I-t segments were recorded after the addition of 52 U L⁻¹ ChOx (Solution C) and 100.0 U L⁻¹ AChE (Solution C).

### Proof-of-Concept #2: Alanine Transaminase (ALT) and Lactate Dehydrogenase (LDH)

$$\begin{matrix}\frac{L \text{-} Alanine + a \text{-} Ketoglutaric Acid {\to }^{•} ALT}{PLP} Pyrurate + L \text{-} Glutamine & ( 33 ) \\ Pyrurate + NADH {\to }^{LDH} Lactate + {NAD}^{\to } & ( 34 )\end{matrix}$$

FIG. 17 depicts ICECEA current-time traces (E=0.40 V) recorded at a glassy carbon/CNT film electrode in a stirred solution of 100.0 mM alanine, 10.0 mM ketoglutaric acid, 1.0 mM pyridoxal phosphate (PLP), and 0.20 mM pyruvate in 0.10 M Tris buffer (Solution A, pH 7.40, 21° C.). The calibration I-t steps are due to the additions of 40.0 μM aliquots of NADH (Solution B). The angled I-t segments were recorded after the addition of 600.0 U L⁻¹ LDH (Solution C) and 100.0 U L⁻¹ ALT (Solution C).

Based on the foregoing data, numerous other enzyme systems can be analyzed via ICECEA including:

### Hexokinase/Glucose-6-Phosphate Dehydrogenase (Hex/G6pDH)

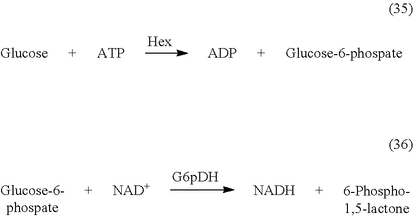

### Superoxide Dismutase (SOD) (Xanthine Oxidase, XOx-Based Generation of Superoxide Radical)

$$\begin{matrix} Xanthine + {O}_{2} + {H}_{2} O {\to }^{XOx} Uric Acid + \text{?} + \text{?} \\ \text{?} + {O}_{2} + {H}_{2} {O}_{2} & ( 37 ) \\ {H}_{2} {O}_{2} \text{?} \\ \text{?} \text{indicates text missing or illegible when filed} & ( 38 )\end{matrix}$$

### Glucose 6-Phosphate Dehydrogenase

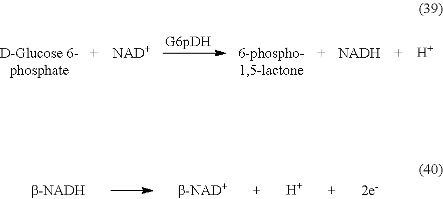

### Glycerol 3-Phosphate Dehydrogenase

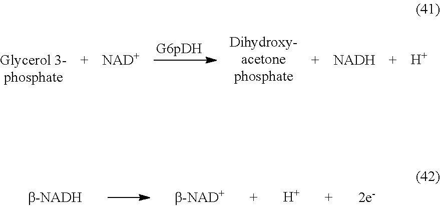

### Esterase

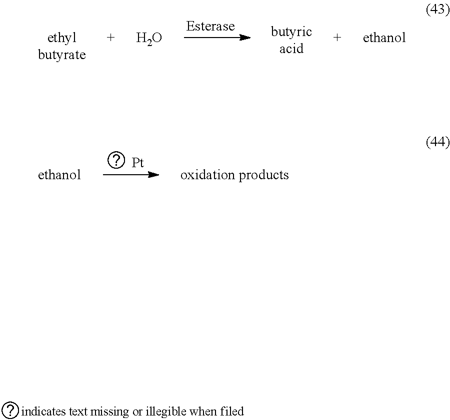

### α-Amylase

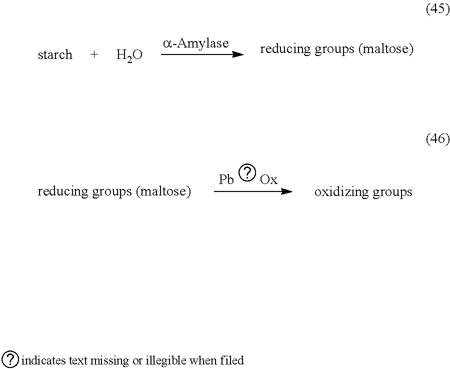

### L-Ascorbate Oxidase (AOx)

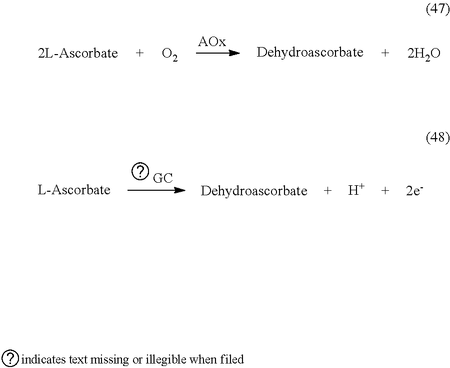

### Glyceraldehyde 3-Phosphate Dehydrogenase (GAPDH)—Use Other Aldehydes as Substrates?

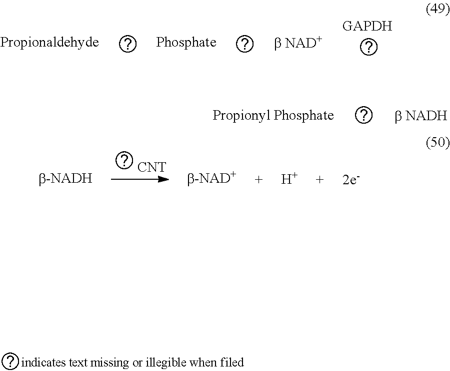

### Alkaline Phosphatase (ALP)

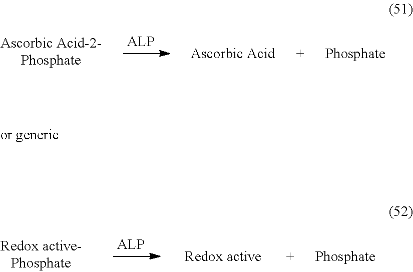

### Galactose oxidase (GalOx)

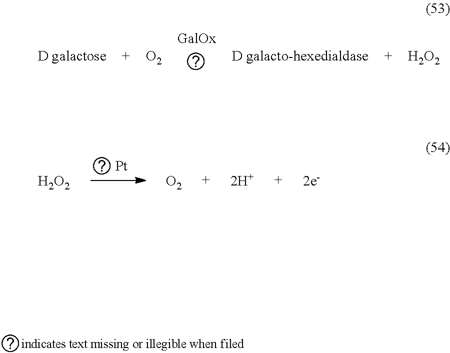

### Aldehyde dehydrogenase (AldDH)

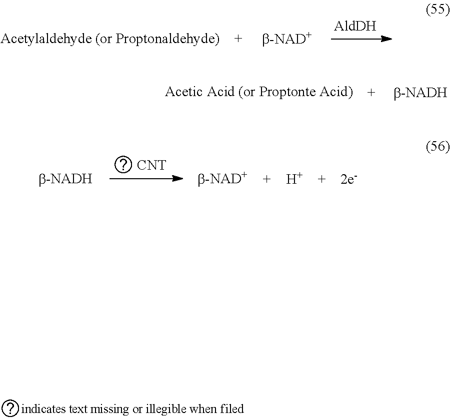

### Other Examples of Enzymes Amenable to Direct ICECEA Analysis

1. Class EC 2 (Transferases)
Enzyme: Adenylyltransferase (EC 2.7.7.2)
Reaction: ATP+FMN⇄diphosphate+FAD
Signal generation: Reduction of FAD at carbon nanotubes

2. Class EC 3 (Hydrolases)
Enzyme: Alpha-amino-acid esterase (EC 3.1.1.43)
Reaction: alpha-amino acid ester+H₂O⇄an alpha-amino acid+alcohol
Signal generation: Oxidation of redox active amino acid at a glassy carbon or oxidation of alcohol at a platinum electrode

3. Class EC 4 (Lyases)
Enzyme: Tyrosine decarboxylase (EC 4.1.1.25)
Reaction: L-tyrosine⇄tyramine+CO₂
Signal generation: Oxidation of L-tyrosine at carbon nanotubes

4. Class EC 5 (Isomerases)
Enzyme: Chalcone isomerase (EC 5.5.1.6)
Reaction: chalcone⇄a flavanone
Signal generation: Reduction of either chalcone or flavanone at a modified electrode

5. Class EC 6 (Ligases)
Enzyme: Tyrosine-tRNA ligase (EC 6.1.1.1)
Reaction: ATP+L-tyrosine+RNA⇄AMP+triphosphate+L-tyrosyl-tRNATyr
Signal generation: Oxidation of L-tyrosine at carbon nanotubes

### ICECEA-Based System for the Determination of Enzyme Activity

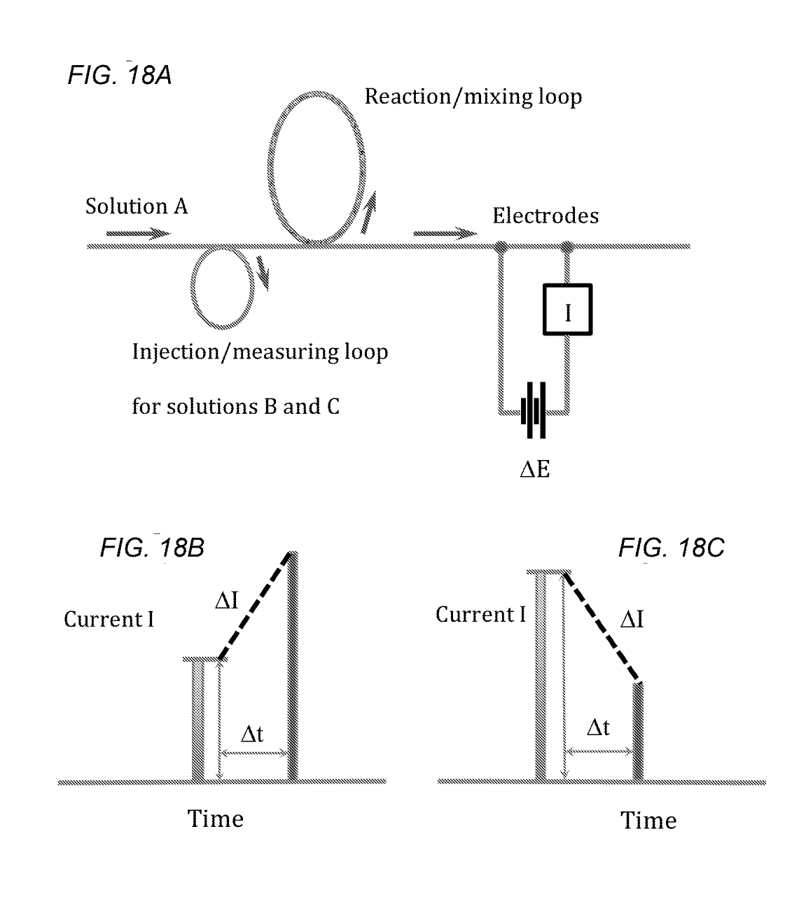

The system uses three solutions: (A) background electrolyte solution containing enzyme's substrate and other necessary reagents, (B) calibration solution containing the redox active component of enzymatic reaction dissolved in the solution A, and (C) background electrolyte solution containing assayed enzyme. The ICECEA-based system includes a flow injection device based on the miniature fluidics in which a solution A is forced to flow through a narrow channel. The system is composed of three parts (FIG. 18A). First, there is the injection/measuring loop that is used to inject well-defined quantities of the calibration Solution B and enzyme Solution C into the flowing Solution A. The injection/measuring loop may be a standard injection loop, such as a loop injector from an HPLC system. A loop injector has at least two configurations. In the first (or loading) configuration, liquid can be introduced into the loop injector while the loop injector is isolated from the flow channel. When the composition is ready to be injected into the flow channel, the injector is changed to a second (or injection) configuration. In the second configuration, fluid flowing through the flow channel is directed into the predefined volume of the injection loop carrying the composition placed in the injection loop into the flow channel.

A reaction/mixing loop is coupled to the flow channel downstream from the injection loop. In the reaction/mixing loop the solutions from the injection loop are mixed with the baseline composition flowing through the flow channel. The reactants and enzymes are mixed in the reaction/mixing loop allowing the enzymatic reaction to proceed for a well-defined time that is dependent on the length/diameter of the loop and flow rate. The reaction/mixing loop can be isolated from the flow channel when not needed. For example, when the calibration Solution B is added to the flow channel, there is no need to delay the flow of the mixed solution to the electrochemical measurement device. When the Solution C is added to Solution A, in some embodiments, a delay may be needed to allow the enzymatic reaction to take place. Thus, the reaction/mixing loop can be switched into the flow channel path, such that the combined Solution C and Solution A is delayed from reaching the electrochemical measurement system.

An electrochemical measurement device is positioned downstream from the reaction/mixing loop. The electrochemical measurement device includes two (or three) electrodes that are poised at a selected potential difference AE and serve to detect the formation or consumption of redox species during the enzymatic reaction.

The system operates as follows. The background Solution A flows through the flow channel. Aliquot of calibration Solution B is injected into the system, via the injection loop, and travels to the electrodes where it generates the calibration current peak (the blue peak in the part FIG. 18B and FIG. 18C). Next, the enzyme Solution C is injected, via the injection loop and is mixed with the Solution A inside the reaction/mixing loop. The reaction mixture A+C is subsequently delivered to the electrodes where it generates the assay current peak (the red peak in the FIG. 18B and FIG. 18C). The assay peak is either larger or lower than the calibration peak depending on what is reacting with the electrodes. For example, the assay peak is larger when electrodes detect the product formed in the enzymatic reaction. On the other hand, the assay peak is smaller if the electrodes detect the reactant that is consumed in the enzymatic reaction. The slope AI/At (FIG. 18B and FIG. 18C) when divided by the calibration slope (I/Concentration) yields the catalytic activity of enzyme.

### CONCLUSIONS

The internally calibrated electrochemical continuous enzyme assay (ICECEA) is a simple, reliable, and cost-effective method for the rapid determination of catalytic activity of a variety of enzymes. The new method does not use enzymes, sometimes fairly expensive, to calibrate the assay. It quantifies the assay via the enzyme-free calibration in the assay solution. This eliminates the need for transferring the working electrode between the calibration and assay solutions, which can affect the activity of electrode surface and lead to erroneous results. The other advantages include no need for extra reagents such as auxiliary enzymes or toxic chromogenic agents. It also does not require the re-polishing of electrodes between assays.

The ICECEA is amenable to automation and miniaturization and is well suited for the applications in bioanalytical and biotechnology fields including the fast analysis of commercial batches of enzymes, quantification of enzyme biomarkers for various diseases, optimization of assays for newly discovered enzymes, and quick corroboration of high-throughput enzyme assays. The combination of time-independent calibration signal with a time-dependent assay signal in one amperometric trace greatly improves the selectivity of ICECEA. This feature can also be explored in the rapid quantitative screening for potential interfering species, enzyme inhibitors, and enzyme substrates.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A method of quantifying enzyme activity comprising:
placing a first composition in an electrochemical assay system, wherein the first composition comprises a substrate of an enzyme;
adding a second composition to the first composition in the electrochemical assay system to create a first assay mixture, wherein the second composition comprises (a) a reactant or product of an enzymatic reaction of the enzyme, or (b) enzyme;
measuring a current flowing through an electrode of the electrochemical assay system after the first assay mixture is formed;
adding a third composition to the first assay mixture to create a second assay mixture, the third composition comprising the enzyme if a second composition above was (a), or a reactant or product of an enzymatic reaction of the enzyme if a second composition above was (b);
measuring a current flowing through an electrode of the electrochemical assay system after the second assay mixture is formed over a predetermined time period; and
determining the enzyme activity based on the change in current over time caused by the addition of the composition containing the enzyme.

2. The method of claim 1, further comprising measuring the changes in current by collecting an amperometric current trace or other electrochemical signal vs. time plots

3. The method of claim 1 or 2, wherein the first composition comprises a background electrolyte.

4. The method of any one of claims 1-3, wherein adding the second composition to the first composition in the electrochemical assay system comprises:
adding a first aliquot of the second composition to the first composition;
measuring a current flowing through an electrode of the electrochemical assay system after the first aliquot is added;
adding one or more additional aliquots of the second composition to the first aliquot/first composition mixture;
measuring a current flowing through an electrode of the electrochemical assay system after each additional aliquot is added.

5. The method of any one of claims 1-4, wherein the enzymatic activity is determined from the slope of a line created from measuring the current passing through an electrode of the electrochemical assay system after the assay mixture containing the enzyme is formed over a predetermined time period.

6. The method of any one of claims 1-5, wherein the addition of the second composition to the first composition and the addition of the third composition to the first assay mixture are performed in the same container using the same electrode.

7. The method of any one of claims 1-6, wherein the enzymes are biomarkers for a disease.

8. The method of any one of claims 1-7, wherein the enzyme activity is quantified in a direct assay.

9. The method of any one of claims 1-7, wherein the enzyme activity is quantified in using a coupled assay.

10. The method of any one of claims 1-7, wherein the activity of the enzyme is quantified using a coupled assay, wherein the coupled assay is represented by the equations:
$$substrate 1 + reactant ( s ) 1 {\to }^{enzyme 1} product 1$$
$$product 1 + reactant ( s ) 2 {\to }^{enzyme 2} product 2$$
wherein the first composition comprises substrate 1;
wherein the second composition comprises (a) reactant(s) 2 or product (2) of an enzymatic reaction of the enzyme 2;
wherein the third composition comprises enzyme 2; and
determining the enzyme activity of enzyme 1 based on the change in current over time caused by the addition of the third composition.

11. An electrochemical assay system configured to perform the method of any one of claims 1-10, comprising:
a flow channel;
an injection loop coupled to the flow channel, wherein the injection loop has a predefined volume;
a reaction loop coupled to the flow channel, downstream from the injection loop; and
an electrochemical measurement device coupled to the flow channel downstream from the reaction loop;
wherein, during use, the first composition flows through the flow channel and the second and third compositions combine with the first composition when the injection loop is opened to the flow channel; and
wherein the reaction loop delays the time that the combined compositions reach the electrochemical measurement device.

12. The system of claim 11, wherein the electrochemical measurement device comprises three electrodes (a working electrode, a reference electrode and an auxiliary electrode).

13. The system of claim 11, wherein the electrochemical assay system comprises two electrodes (a working electrode and a reference electrode).

14. The system of claim 12 or 13, wherein the working electrode comprises a noble metal electrode, an electrode made of a carbon allotrope, or a modified electrode.

15. The system of claim 12 or 13, wherein the auxiliary electrode comprises platinum.

16. The system of claim 12 or 13, wherein the reference electrode is a Ag/AgCl/NaCl or any other reference electrode.

17. The system of any one of claims 11-16 wherein the reaction loop comprises a length and/or diameter that creates a predetermined delay between the injection loop and the electrochemical measurement device.

18. A method of quantifying enzyme activity comprising:
placing a first composition in an electrochemical assay system, wherein the first composition comprises a substrate of an enzyme;
adding an enzyme composition to the first composition to create an assay mixture;
measuring a current flowing through an electrode of the electrochemical assay system after the second assay mixture is formed over a predetermined time period.
